(19) United States
(12) Patent Application Publication
(10) Pub. No.: US 2009/0306621 A1
(43) Pub. Date: Dec. 10, 2009

(54) Vial transfer convenience IV kits and methods

(75) Inventors: Gale H. Thome, JR., Bountiful, UT (US); Kendall P. Thome, Layton, UT (US); Gale H. Thome, Bountiful, UT (US)

Correspondence Address: Gale H. Thome, 1056 Millcrest Circle, Bountiful, UT, 84010, US

(73) Assignee: Gale H. Thome, JR., Bountiful, UT (US)

(21) Appl. No.: 12/460,470

(22) Filed: Jul. 20, 2009

Related U.S. Application Data
(63) Continuation-in-part of application No. 12/319,326, filed on Jan. 6, 2009.
Application No. 12/319,326 is a continuation-in-part of application No. 12/313,013, filed on Nov. 14, 2008, now abandoned.
Application No. 12/313,013 is a continuation-in-part of application No. 12/080,185, filed on Apr. 1, 2008.
Application No. 12/080,185 is a continuation-in-part of application No. 12/012,837, filed on Feb. 6, 2008.

(51) Int. Cl. A61M 39/16; A61M 39/18

(52) U.S. Cl. 604/500; 604/82; 604/262

(57) ABSTRACT

Convenience kits designed to provide for closed, but selectable liquid transfer from a vial to a variety of IV containers and medical syringes. In particular, a kit for fully enclosing a vial for safety in hazardous drug transfer is disclosed. Generally, the kits contain unitized parts wherever reasonable to limit makes and breaks. Further, pathway determining kits provide selectable pathways for purging connections wish flushing solution where makes and breaks are made between various fluid pathway involved parts such that, when disconnections are made, flush solution is resident at the exposed interface. Also disclosed is a 3-way valve as part of a closed, switchable pathway controlling subsystem by which pathways are selected for reconstituting dry medicine in a vial, displacing a measured dose of liquid from a vial, exchanging gas into the vial for displaced liquid, delivering the measured dose to an IV container.

### CONTINUATION-IN-PART

This application for patent is a Continuation-in-Part of U.S. patent application Ser. No. 12/319,326 filed Jan. 6, 2009, which is a Continuation-in-Part of U.S. patent application Ser. No. 12/313,013, filed Nov. 14, 2008, now abandoned, which is a Continuation-in-Part of U.S. Ser. No. 12/080,185, filed, Apr. 1, 2008, which is a Continuation-in-Part of U.S. patent application Ser. No. 12/012,837 filed Feb. 6, 2008, the contents of which are made part of this application by reference.

### FIELD OF INVENTION

This invention relates to medical intravenous administration of fluids, specifically for medical applications including push or bolus and drip (from a hanging container) dispensing. It is also particularly related to kits and to methods which employ preassembled parts and which are substantially fabricated for the purpose of achieving a closed system when transferring solutions from vials to IV dispensing systems used in patient drug delivery and especially for drug delivery using medicines which present a hazard if exposed to an open environment.

### BACKGROUND AND DESCRIPTION OF RELATED ART

### A Review of Problems Associated with Hazardous Drug Handling

A 2004 NIOSH (National Institute of Occupational Safety and Health) Safety Alert: Preventing Occupational Exposure to Antineoplastics and Other Hazardous Drugs in Healthcare Settings warns healthcare institutions about the need to provide products and procedures to protect clinicians from hazardous drug exposure. Attempts to reduce such drug exposure has resulted in use of expensive protective port attachment devices.

In applications from which this U.S. patent application continues, generally direct patient IV delivery was addressed. However, instant inventions disclosed and claimed herein generally relate to apparatus and methods associated with Pharmacy procedures for transferring medications from a vial to a vessel for medication delivery to patients via a container which receives the medications from the vial.

While USP Chapter 797 recommends that healthcare workers transferring medication from medical vials draw from single dose vials and while avoiding use of multi-dose vials due to microbial contamination concerns, many facilities still use multidose vials that are associated with chemotherapy and other hazardous drug (HD) agents. Most frequently, the reasons given are associated with the caustic nature of such medications and the common belief that microbes cannot survive within such vials. Cost is certainly a significant driving force. While one may challenge such assertions, the reality is that devices that address multi-dose vial use with an emphasis on clinician and patient safety is essential in most healthcare facilities when chemotherapy agents are involved.

Contemporary closed system safety devices associated with HD transfers, strive to make “dry” connections and disconnections (i.e. to disconnect using cleverly engineered connectors which have a primary goal of leaving no liquid residue exposed at the connection site after disconnection). It is duly noted that none of these types of devices, currently known by Applicants, to date provide a flush prior to disconnection as recommended by NIOSH. It is also duly noted that, from time to time such devices are prone to leakage. Such leakage often results in droplets being left on connecting septa where exposure risk can result from direct contact or secondary contamination as droplets may come in contact with personal protective equipment such as gloves and gowns of primary users or even by others who may inadvertently come in contact with such residues. Further, clinicians have identified a “smear effect” with HDs as multiple connections are made at leakage sites.

Vials containing HDs have been found to have HD contamination on outer surfaces of such vials at the time of receipt by a user. Per institutional protocol, healthcare workers are often instructed to wash HD containing vials before and after use to minimize the spreading of HD residue. There may even be a concern about the spread of HD as a result of the washing process. If a user touches another surface while cleaning, drug may be spread. HD may also be spread as a result of the paraphernalia used in the cleaning process itself. Some drugs require special methods or cleaners to assure that drug is removed vs. being simply spread around.

When considering use of multi-dose vials, users must take into account that many chemotherapy agents are transferred to IV containers (e.g. IV bags) or to syringes for delivery through Y-injection sites and other IV ports. Vials may only provide a portion of a dose, requiring an exchange of vials to prepare a single dose.

In summary, there are basically three sites concerning potential exposure of HD while making a transfer from a vial to a receiving container used in medication delivery. A first site is at the vial itself where the exterior of the vial may be contaminated and where a vial septum, once pierced may emit HD. A second site is at a vial fluid access point (usually through a needleless connector) where the vial may be disconnected from an HD transfer system. The third site is associated with disconnection of a receiving (and delivery) container from a HD transfer system.

Also a basic principle in IV therapy, which must be considered in patient related practices, is that the more connections and disconnections made to an IV line, the greater the probability that that line will become contaminated by microbial agents. The same may also be said relative to HD safety. The more connections and disconnections, the greater the potential for an HD leak or spill. A product which reduces IV line connections and disconnections, then, should provide added safety for both clinicians and patients.

Currently, for every safety HD closed system known to be currently in use by the Applicants, there is a requirement for a user to draw medication, disconnect from a vial adaptor, reconnect to an IV container adapter, inject the medication into the IV container and then disconnect whether the system employs either “dry” or a flush. Therefore, there is a need for a system which allows for a medication to be drawn from a vial, measured accurately and then immediately transferred to an IV container. Such a system is projected to clearly provide a significant user and patient safety advantage and is the character of apparatus, devices and methods of invention disclosed herein.

Because current safety devices are known to leak, and because flushing has been demonstrated to provide a safety “saline-saline”, or other flush solution, disconnection site, thereby reducing the probability of an exposure risk, all apparatus, devices and methods associated with the instant invention herein disclosed employ a flushing facility at each disconnection site. Further, noting that dead space is a critical parameter since dosing accuracy is a concern when preparing HD's, products designed for such preparation should have minimal dead space.

Another concern when transferring fluid from a vial is vial stability. Due to effects of gravity relative to vial orientation for optimal HD drawing conditions, devices must avoid “flopping over” or tilting out of position. It is preferred that a vial transfer system should provide adequate support for the vial to assure that each vial is maintained in a top-down orientation. Further, escaping contaminated gases and aerosols may also pose a serious health-risk. For this reason, pressurizing of vials during fluid transfer is generally discouraged. Stated otherwise, to minimize HD exposure technique of transferring fluid from a vial using negative pressure technique is preferred.

### Terms and Definitions

In the following table 1 is a list of terms and associated definitions provided to improve clarity and understanding of precepts of the instant invention:

TABLE 1
break, n: a disconnection of a pair of medical connectors, as part of a medical procedure.
dead space, n: a volume of inaccessible fluid, retained within a device after a procedure.
dose syringe, n: an initially empty syringe which is filled with a prescribed dose of
medication.
flush syringe, n: a syringe, pre-filled with a predetermined volume of flush solution.
HD, n: hazardous drag
IV set, n: intravenous drug delivery tubing specifically dedicated for use with an
associated IV catheter and IV container.
IV container, n: a container, made of glass or plastic in the form of a bottle or IV bag used
to hold and deliver IV fluids containing a saline solution and/or other medications for
delivery through an IV set to a patient.
kit, n: a group of parts, provided within a single package for a designated medical use
luer fitting, n: a medical connector which is in common use in medical practice.
luer lock fitting, n: a luer fitting having a locking mechanism whereby a male and female
connector are securely, but releasibly affixed one to the other.
make, n: a connection or re-connection of a pair of medical connectors.
multi-dosing, v: action of drawing more than one dose of medication from a single vial
needleless connector, n: a fitting which permits needle free fluid access to an IV set or
through a vial adapter and which has interface geometry similar to a conventional syringe
port, n: a site for a medical connector, where through fluid is communicated
short extension set, n: tubing and associated connecting parts used for connecting a fluid
valve to a pre-filled syringe.
shroud, n: a vial and vial adapter containing cover which provides a barrier against fluids
emitted from a vial septum during fluid acquisition from the vial.
subsystem, n: a part of a system.
unitized, adj: a plurality of separate parts permanently joined to be used as a single unit.
vial, n: a medication container in which medication is delivered from a manufacturer to a
medical facility, fluid in the vial is usually accessed via a spike of a vial adapter which
pierces a septum of exposed diaphragm of the vial.

### BRIEF SUMMARY AND OBJECTS OF THE INVENTION

In brief summary, this novel invention provides a basis for a safety system for transferring medications from a vial to a receiving vessel through a system which is closed during transfer. It also provides for flushing separate-able sites which are disconnected before transport of the vessel to a site of use away from the vial transfer system. Requirement for such a system is commonly found in Pharmacy, especially for those instances where hazardous drugs are involved.

Generally, the instant invention disclosed herein is embodied in convenience kit products which are combinations of standard, selected components which have been proved and certified for medical use. In most instances, the invention is embodied in two subsystems, a vial handling and accessing subsystem and a fluid pathway controlling subsystem. While the two subsystems may be combined and fabricated as a single joined system, it is most often preferred to provide the two subsystems as separate kits. Also, in a preferred embodiment, the two subsystems may be separated after use and after flushing each site where the two subsystems are interconnected.

The first subsystem provides a shroud into which a drug containing vial may be displaced and enclosed for safety in handling and accessing. The shroud is preferably made from material which is impervious to gases and other matter which may be on or emitted from the vial when the vial septum is punctured. Further, shroud material should have sufficient strength to fully support an enclosed fully filled vial but being sufficiently pliant to permit manual external handling of parts within the shroud. Also contained in the shroud is a portion of a vial adapter comprising a vial adapter spike. Commonly, the vial adapter spike is supplied with a protective cover which protects sterility of the spike and from unintended and undesired piercing of surrounding items prior to insertion of the spike into the vial septum. The outflow portion of the vial adapter is disposed through a barrier at a supporting bottom section of the shroud to provide fluid flow access outside the shroud through the outflow portion of the vial adapter. It is preferred to attach a needleless connector to the outflow portion of the vial adapter to provide a detachable connecting site whereat the second subsystem can be releasibly affixed for reasons disclosed in detail hereafter. Note that the needleless connector should be selected for ability to be purged by flushing and containment of liquid when unattached.

The shroud may be made from a plastic bag, similar to a sandwich bag, but preferably of heavier material (e.g. 2 mil thickness) to provide a barrier for safer handling and covering. Similar to a sandwich bag, such a shroud may have a zipper closure, although other closures, such as a constrictive simple tie, may also be used. A hole, for example, in shroud material superiorly disposed to the closure is preferred to permit the shroud to be hung and provide for hand-free operation, freeing hands for other procedures during fluid transfer. It should be noted that hanging such a shroud provides substantial support to a vial affixed to a vial adapter affixed to the bottom or inferior portion of the shroud.

Preferably, the second subsystem is interconnected to the first subsystem through a needleless connector. The second subsystem provides for flow path selective switching and therefore pathway control of flow of fluids involved in transferring medication from the vial to a measuring instrument and therefrom to a liquid receiving container. Where fluid volume transferred makes it expedient to infuse gas into a vial to replace displaced dispensed liquid to reduce accrued negative pressure inside the vial to a desired amount, the second subsystem provides a first open pathway either for directly transferring gas into the vial or for transferring gas into the vial via the measuring instrument. In this latter case, gas is displaced into the measuring instrument thru a pathway opened from a gas source (e.g. open to atmosphere, preferably through a filter). Once sufficient gas is displaced into the measuring instrument, a second pathway is opened from the measuring instrument to the vial and liquid is drawn from the vial while replacement gas is substituted therefor. It should be noted that, even though the vial is disposed in a protective shroud, negative pressure liquid transfer technique is recommended.

When filling an IV vessel or container after a predetermined volume of liquid (e.g. a dose of medication) has been drawn from the vial into the measuring instrument, a third pathway is opened to permit delivery of liquid from the measuring instrument to the receiving vessel or container. However, at this stage, all connecting sites (i.e. between the first and second subsystems and between the second subsystem and receiving vessel or container) are contaminated with liquid or medication from the vial.

To achieve safety in disconnecting one subsystem from the other or the second subsystem from the receiving vessel or container, adequate flushing of the connecting sites is provided via pathway control within the second subsystem. For this purpose, the second subsystem also provides a source of flush solution and pathway selecting apparatus, which can be switched, to selectively provide a pathway for fluid displacement through a connecting site between the second subsystem and the receiving vessel or container. If it is desired to disconnect the two subsystems, the second subsystem also provides a selectable pathway for flush solution to the needleless connector of the vial access subsystem. Note that the system is closed during medication transfer with connecting sites only opened after flushing to assure only flush liquid is exposed upon disconnection.

Exemplary procedures for using such a system involves:
a. providing the desired convenience kits;
b. assembling the kits into a closed vial transfer system;
c. following institutional protocol for handling a selected vial, displacing the selected vial into the shroud and enclosing and sealing the vial within the shroud;
d. as needed, removing any protective covering from the spike of the vial adapter and perforating a pierceable septum of the vial with the spike whereby fluid within the vial is made accessible through the vial adapter without direct manual contact with vial adapter or vial;
e. preferably using negative pressure technique, drawing a predetermined volume of liquid from the vial into the measurement instrument;
f. dispensing the predetermined volume of liquid from the measurement instrument into a receiving container;
g. providing a communicating pathway between the source of flushing liquid and the exterior connector through which liquid was dispensed to the liquid receiving container; and
h. flushing through the exterior connector and thereafter disconnecting the exterior connection associated with the exterior connector to thereby only expose flushing solution to the environment exterior to the otherwise closed system.
Other steps which are associated with system operation include:
1. If it is desired to separate the two subsystems, open a pathway between the source of flushing liquid and the needleless connector and flush before separating the two subsystems to thereby only expose flushing solution to the environment exterior to the otherwise closed system.
2. Selecting a syringe, which may be purchased and provided by the institution rather than being a kit part, for providing a measurement instrument and a pre-filled flush syringe.

Accordingly, it is a primary object to provide methods and apparatus for preparing and using convenience kits for closed system transfer of medications from a vial to a patient IV dispensing device.

It is a principal object to provide a convenience kit which affords a closed system for such transfer, yet permits separation between the vial and patient dispensing device without exposing medication to the environment.

It is a major object to provide a discardable convenience kit which supplies an enclosing shroud for a vial which protects during medication transfer and provides an enclosing shield for disposal

It is a primary object to provide a vial transfer convenience kit which provides that fluids acquired from a source vial and transferred to a patient IV dispensing system be kept in a closed environment through transfer and further provides for flushing of any connection to be opened after such transfer prior to opening or disconnecting such that there is only a flush solution (e.g. saline-saline) interface at the sites of disconnection.

It is an object to provide a handling shield for the exterior of a vial during transfer of medication from a vial.

It is an object to provide for stabilizing support of a vial affixed to a vial adapter.

It is an object to provide for affixing a vial adapter to a vial in an environment which provides a barrier to fluids which may be emitted from an associated vial when attaching the vial adapter to the vial and which may be emitted as part of a process of transferring liquid from the vial.

It is an object to provide for hanging an enclosing shield about a vial adapter such that hands otherwise used for support of the vial are freed to be used for other medication transfer purposes.

It is a very important object to provide apparatus for controlling gas replacement of liquid drained from a vial.

It is a another very important object to provide for a predetermined disposal path for flushing fluids through each disconnectable site.

It is yet another very important object to provide for maintaining a source of flushing fluids in an uncontaminated state by medication acquired from a vial.

It is an object to provide a plurality of convenience kits which provide transfer capability which ranges from medication transfer to hand held syringe delivery and flushing devices to IV containers including bags and elastomeric balls, such kits having a commonality of function of providing closed system transfer, a protective shield about vials, controlled pathways for medicine and other fluid flow and flushing of separable connections.

It is a consequential object to provide a convenience kit having a closed, switchable pathway controlling subsystem which further provides for a communicating pathway through a stopcock having a needleless connector compatible male luer connector interface to provide a connecting site with a needleless connector which provides fluid access to a vial adapter and there through to a vial for displacing fluids to and from the vial, for a first selectable pathway through which liquid is introduced for the purpose of reconstituting dry medicine in a connected vial and for the purpose of flushing the connecting site prior to disconnecting the needleless connector compatible luer connector interface from the needleless connector and through which gas is exchanged for liquid drawn from the vial, for another pathway which provides fluid communication between the vial and a measuring instrument whereby a predetermined volume of liquid is displaced from the vial, for still another pathway where through the predetermined volume of liquid is displaced to a receiving IV container through a connecting delivery site at which the IV container is affixed to be filled and for still another pathway for flushing the connecting delivery site prior to disconnecting the closed, switchable pathway controlling system from the receiving IV container for delivery to a site of use.

It is a critical object of kits made according to invention disclosed in this application that kitted adjoined parts be unreleasibly affixed (unitized) to preclude separation in transport and storage.

These and other objects and features of the present invention will be apparent from the detailed description taken with reference to accompanying drawings.

### BRIEF DESCRIPTION OF THE DRAWINGS

FIG. 1 is a perspective of a dual syringe assembly, comprising two syringes, a stopcock and a syringe clip, configured to provide a dose dispensing and flushing apparatus.

FIG. 1A is a perspective of a stopcock and an associated short extension set portion of the assembly seen in FIG. 1.

FIG. 1B is a magnified perspective of the stopcock and a portion of the associated extension set seen in FIG. 4C.

FIG. 1C is a front elevation of a PRIOR ART stopcock.

FIG. 1D is a front elevation of a stopcock having a male luer fitting made in accordance with the present invention.

FIG. 2 is a perspective of an assembly which is similar to the dual syringe assembly seen in FIG. 1, but which incorporates a pressure operated fluid switch assembly in place of the stopcock as seen in FIG. 1.

FIG. 3 is a schematic layout of parts for a vial adapter kit made according to the present invention, the kit comprising a bag which may be resealed at a superior end, a vial adapter and barrier providing parts and a needleless connector.

FIG. 4 is a schematic layout of parts, seen in FIG. 3, assembled to provide a vial adapter system convenience kit.

FIG. 5 is a schematic layout of a vial being inserted into the bag seen in FIGS. 3 and 4.

FIG. 6 is a schematic layout of the vial, seen in FIG. 5, enclosed within the bag.

FIG. 7 is a schematic layout of the vial, seen in FIG. 6, affixed to a spike of the vial adapter, following removal of a vial adapter spike cover or guard from the spike.

FIG. 8 is an exploded schematic layout of parts associated with a fluid pathway-determining and selecting convenience kit designed for use with a vial adapter assembly.

FIG. 9 is a schematic layout of an assembled fluid pathway determining and selecting convenience kit in which parts seen in FIG. 8 are joined.

FIG. 10 is an exploded view of a plurality of assembled convenience kits selected for fluid transfer between a vial and a dual syringe assembly seen in FIG. 1.

FIG. 11 is a schematic of the convenience kits seen in FIG. 10 interconnected for use.

FIG. 12 is an exploded schematic layout of parts associated with a fluid pathway determining and selecting convenience kit designed for filling a receiving container (for example an IV bag or elastomeric ball which may be used for delivery through a patient IV set).

FIG. 13 is a schematic layout of an assembled fluid pathway determining and selecting convenience kit in which parts seen in FIG. 12 are joined.

FIG. 14 is an exploded schematic view of a plurality of assembled convenience kits, including the kit seen in FIG. 13, selected for fluid transfer between a vial and a fluid delivery tube which may be connected to a receiving container.

FIG. 15 is a schematic of the convenience kits seen in FIG. 14 interconnected for use.

FIG. 16 is a schematic of the interconnected parts seen in FIG. 15 connected to a receiving container.

FIG. 17 is a schematic layout of parts for a vial adapter kit which are similar to parts seen in FIG. 3 except for the vial adapter, the vial adapter in this case being a vented vial adapter.

FIG. 18 is a schematic layout of parts, seen in FIG. 17, assembled to provide a vial adapter system convenience kit.

FIG. 19 is an exploded schematic layout of parts associated with a fluid pathway determining and selecting convenience kit designed for use with a vial adapter assembly and to provide a sequential medication delivery system.

FIG. 20 is a schematic layout of an assembled fluid pathway determining and selecting convenience kit in which parts seen in FIG. 19 are unitized.

FIG. 21 is an exploded schematic view of a plurality of assembled convenience kits and associated syringes for the purpose of selectively providing for fluid transfer between a vial and a plurality of syringes.

FIG. 22 is a schematic view of convenience kits and associated syringes joined for use.

FIG. 23 is an exploded schematic layout of parts associated with a fluid pathway determining and selecting convenience kit employing a single 3-way stopcock and designed for use with a vial adapter assembly to provide a sequential medication delivery system.

FIG. 23A is a schematic view of a 3-way stopcock showing a first port closed.

FIG. 23B is a schematic view of a 3-way stopcock showing a second port closed.

FIG. 23C is a schematic view of a 3-way stopcock showing a third port closed.

FIG. 24 is a schematic layout of an assembled fluid pathway determining and selecting convenience kit in which parts seen in FIG. 23 are joined.

FIG. 25 is an exploded schematic view of a plurality of assembled convenience kits and associated syringes for the purpose of selectively providing for fluid transfer between a vial and an IV receiving and delivery container.

FIG. 26 is a schematic view of convenience kits and associated syringes joined for use.

FIG. 27 is a schematic of the interconnected parts seen in FIG. 26 connected to a receiving container.

### DETAILED DESCRIPTION OF THE ILLUSTRATED EMBODIMENTS

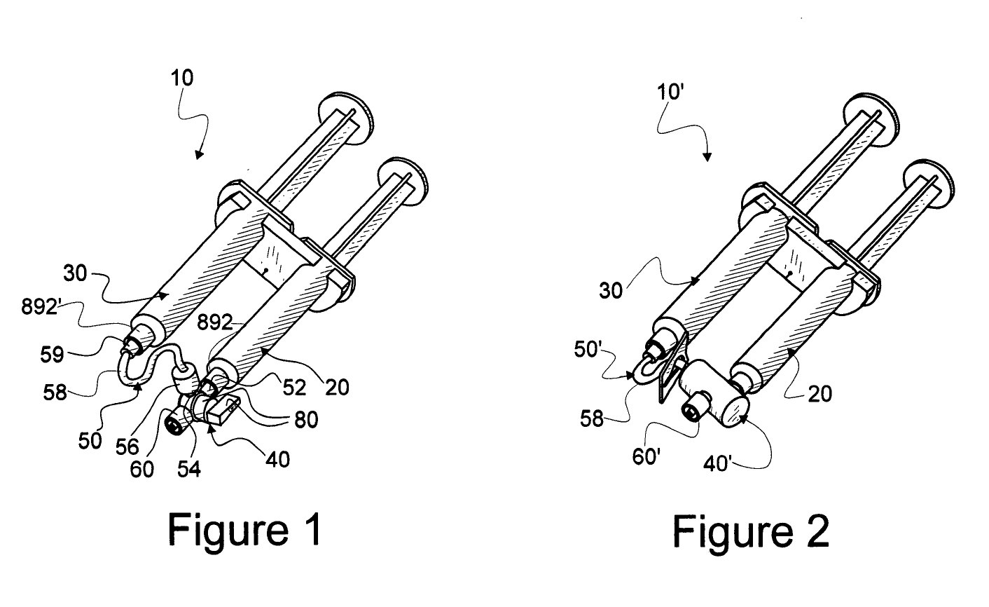

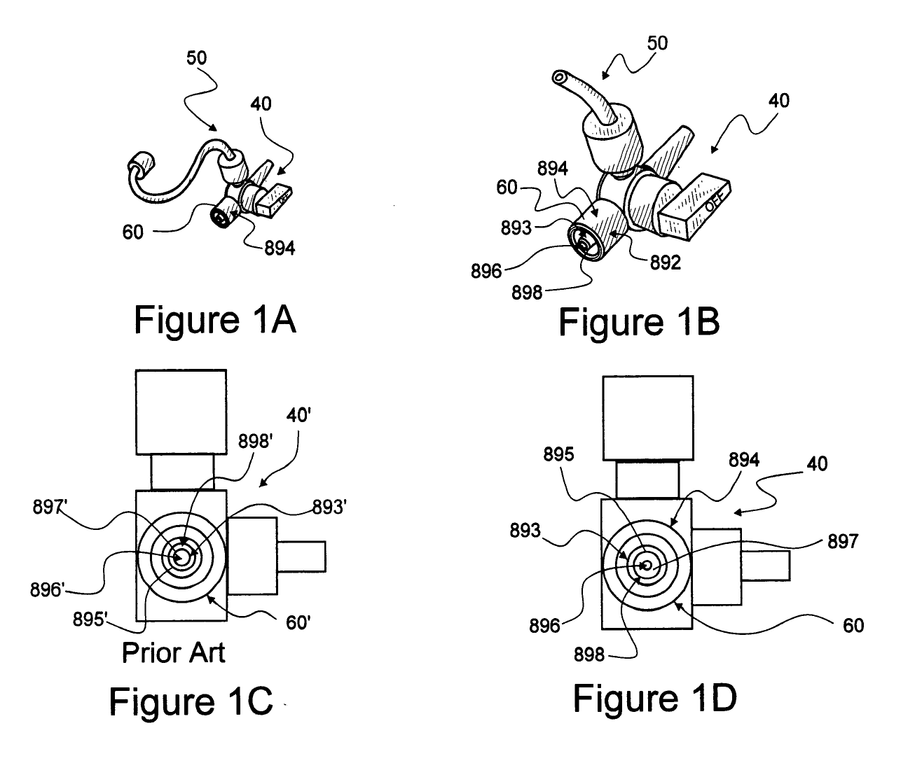

In this description, the term “proximal” indicates the segment of the device normally closest to the object of the sentence describing its position. The term distal refers to a segment oppositely disposed. Reference is now made to the embodiments illustrated in FIGS. 1-27 wherein like numerals are used to designate like parts throughout. For parts which are similar but not the same as parts originally specified with a given number, a prime of the original numbers is used. It is important that all parts selected for use in convenience kits associated with the instant invention, be able to be sterilized, for example, by such methods as gamma radiation.

Reference is now made to FIG. 1 wherein a first convenience kit assembly 10 is seen to be readied for dispensing of fluids from a pair of syringes, numbered 20 and 30. It should be noted that characteristics and details of assembly 10 are fully disclosed in U.S. patent applications from which this U.S. patent application continues in part. Relative to a user, syringes 20 and 30 are distally interconnected through a stopcock 40 and micro-bore tubing (short extension) set 50. Each syringe 20 and 30 may be a conventional commercially available medical syringe. One syringe, in particular syringe 30, may be a commercially available pre-filled flush syringe.

Stopcock 40 has three ports, a first port 52 being a female, preferably luer lock, connector which is securely affixed to syringe 20; a second port 54 also being a female, preferably luer lock, connector for connecting to a male connecting port 56 of a tubing set 50. At an opposite end, tubing set 50 has a female, preferably luer lock fitting 59 for secure attachment to syringe 30. Note that port 54 of stopcock 40 is disposed at right angles relative to port 52. Compliance and flexibility of tubing 58 of tubing set 50 permit syringe 30 to be aligned with syringe 20 for common operation with a single hand. A male, preferably luer lock, fitting 60 is exposed for attachment to a port, e.g. an injection port or a vial adapter, where through fluid is communicated. It is important that fitting 60 is compatible with needleless connectors.

Fluid flow from assembly 10 is controlled by position of rotation of a core and handle 80 of stopcock 40. As seen in FIG. 1, fluid communication into and from syringe 20 is obstructed by the position of core and handle 80. Rotation of core and handle 80 to a position obstructing outflow from syringe 30 opens outflow from syringe 20 to controllably permit selective dispensing of fluids from syringes 20 and 30 while keeping fluids within syringes 20 and 30 disparate. It is emphasized that use of a stopcock to control fluid flow is not the only way for such control; however, a stopcock as disclosed herein provides an efficacious way of dealing with requirements for keeping fluids separate in syringes 20 and 30.

While use of a stopcock, such as stopcock 40, is in accord with the first kit, an alternative, which requires no external manual switching is provided by a pressure actuated fluid switching apparatus 40′, seen in FIG. 2 and disclosed in detail in U.S. patent applications from which this application continues-in-part. As seen in FIG. 2, apparatus 10′ is affixed to a dose syringe 20 and a flush syringe 30 in the same manner that dose and flush syringes are affixed to a stopcock 40. Interposed between flush syringe 30 and a pressure actuated switching apparatus 40′ is a length of micro-bore tubing 58 which is part of a short extension set 50′ which is similar in form and function to set 50, disclosed supra. In the case of apparatus 10′, each syringe 20 and 30 may be a conventional commercially available medical syringe. Apparatus 10′ simply permits bidirectional fluid flow to and from syringe 20 to a port connecting fitting 60′ and unidirectional flow from syringe 30 to fitting 60′ while keeping fluids in syringes 20 and 30 disparate.

While previously filed patent applications from which this application continues in part were focused upon preparation and uses for assemblies 10 and 10′, this present disclosure concentrates on handling and filling apparatus and procedures mainly for use in a pharmacy. The general problem addressed is fluid transfer from a vial to a container which delivers patient medication, keeping such an apparatus closed during such transfer and, only after transfer is completed, flushing any site where a disconnection is afterward made to assure fluids exposed at the disconnection site are substantially flushing liquids.

Fluid Access from a Vial (a Vial Access Convenience Kit)

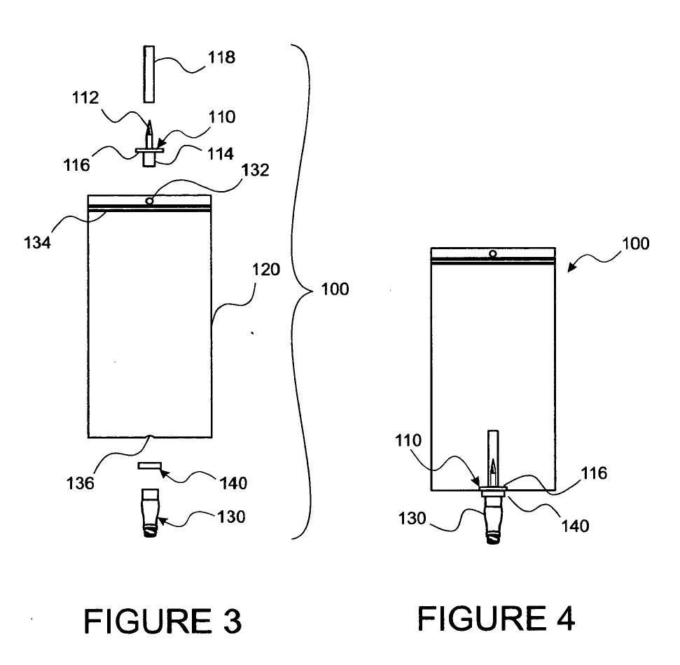

Parts for a vial access convenience kit 100 made according to the present invention are seen in FIG. 3. Kit 100 includes a vial adapter 110 having a vial adapter spike 112, a dispensing port 114, a flange stop 116 and a spike cover 118, a vial shrouding bag 120 (preferably made from a material which is clear and pliant but having sufficient thickness and durability to bear downward forces disposed upon a vial without tearing), and a valved component 130, such as a needleless connector which is closed to fluid flow until interconnected to a communicating fitting (such as a luer fitting).

For purposes which shall be disclosed in detail hereafter, bag 120 preferably comprises a hole 132 from which bag 120 may be suspended from a hook or the like, a zipper seal 134 inferiorly disposed to hole 132 and a second hole 136 through which adapter 110 dispensing port 114 may be displaced to provide a fluid access pathway out of vial shrouding bag 120. Further kit 100 comprises a barrier or gasket part 140.

Preferably, parts of kit 100 are unitized in the following manner to provide an assembled kit 100 as seen in FIG. 4. Preferably, barrier or gasket part 140 has sufficient thickness to provide a compressive fit between needleless connector 130 and connector stop 116 to provide a tortuous path barrier there between. Vial adapter 110 is inserted into vial shrouding bag 120 until dispensing port 114 is displaced through hole 136. Gasket part 140 is displaced about the so exposed dispensing port 114 and needleless connector is preferably securely affixed and adhesively affixed to dispensing port 114. As is well known to those skilled in medical packaging, kit 100 is then packaged in a sealed package, labeled and sterilized prior to transport and use, as in the case for all convenience kits associated with this disclosure.

Vial adapter 110 may be a Medegen Vial Adapter, part number MV0510. Bag 120 may be a polyethylene medical grade bag available from Discount Plastic Bags, LLC, part number F20408H. Needleless Connector 130 may be a needleless connector available from ICU Medical, Cardinal Alaris or Halkey Roberts. These needleless connectors are cited as they have been tested to be successfully cleared by flushing by Applicants. Gasket part 140 may be made from a closed cell foam material or low durometer butyl rubber which does not particulate and should be of sufficient thickness to provide a compressive fit.

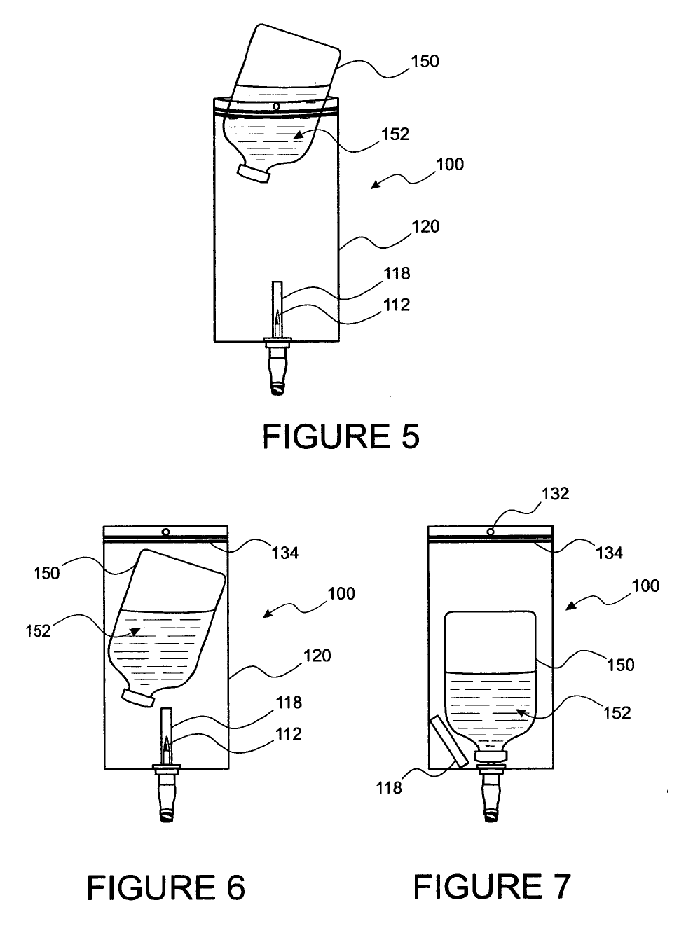

Reference is now made to FIGS. 5-7 wherein illustration for enclosing a vial (generally numbered 150) is provided. As seen in FIG. 5, vial 150 (with a pierceable septum exposed for penetration) is displaced through an opening at the top of shrouding bag 120. Vial 150 is generally provided as a source of medicine 152 for transfer to a patient medical delivery assembly or device. Note, that it may be preferable to set vial 150 upright on a flat surface and displace vial shrouding bag 120 downward about vial 150 rather than displacing vial 150 downward as seen in FIG. 5. Once vial 150 is disposed within and shrouded by bag 120, as seen in FIG. 6, zipper seal 134 is closed. Of course, there are other bag types which may be used, such as bags which may be closed by a tie and serve well as a shroud for vial 150.

With vial 150 enclosed in shrouding bag 120, spike cover 118 is removed from spike 112 and the septum (not shown) of vial 150 is pierced by spike 118. Note that spike cover 118 may be removed and vial 150 spiked without direct glove or hand contact of so enclosed vial 150. Also the tortuous path and compressive barrier provided by barrier or gasket part 140 and zipper seal 134 adds to the safety of enclosure.

### Syringe Filling Convenience Kit

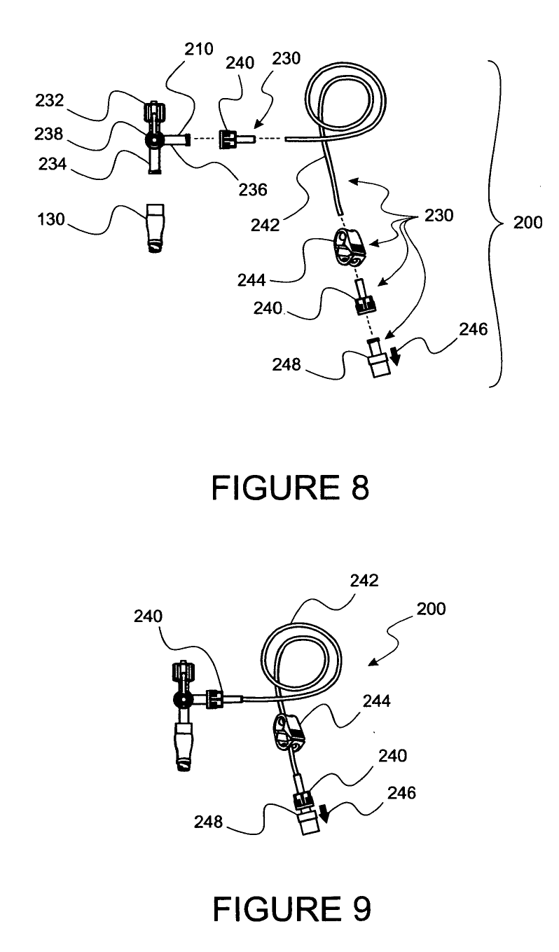

For filling assemblies 10 and 10′ (see FIGS. 1 and 2), a fluid pathway selecting kit 200 provides for necessary pathways and pathway switching as seen in FIGS. 8-11. Parts of Kit 200, seen in exploded format in FIG. 8 comprise a 2 way stopcock 210, a needleless connector 130, and micro-bore tube extension set 230.

Stopcock 210 has a male luer lock, needleless compatible fitting 232, a female luer fitting 234 in line with fitting 232 and a female luer fitting 236 orthogonally disposed relative to fittings 232 and 234. Further, stopcock 210 has a rotatable turn mechanism 238 which when rotated to a first position closes a pathway through fitting 232 (as seen as disposed in FIG. 8) to provide an open fluid pathway between fitting 234 and 236. When mechanism 238 is rotated to a second position (not shown) to close a pathway through fitting 236, a pathway between fitting 234 and 232 is opened. As mentioned supra, needleless connector should be readily cleared by flushing and may be a needleless connector 130 available from ICU Medical, Cardinal Alaris or Halkey Roberts.

Micro-bore tubing extension set 230 comprises a first male luer-to-tube fitting 240 disposed proximally at fitting 236, a length (preferably about 15 inches) of micro-bore tubing, a tubing clamp 244, a second male luer-to-tube fitting 240, a one way (check) valve 248, having an open direction of flow as defined by arrow 246. All parts of set 230 are well known and available in the medical assembly fabrication art and will not be further defined herein.

All parts of the assembly associated with kit 200, with the exception of clamp 244, are assembled and adhesively joined as seen in FIG. 9. Clamp 244, is not adhesed, but is captured about micro-bore tubing 242 and between first and second male luer fittings 240.

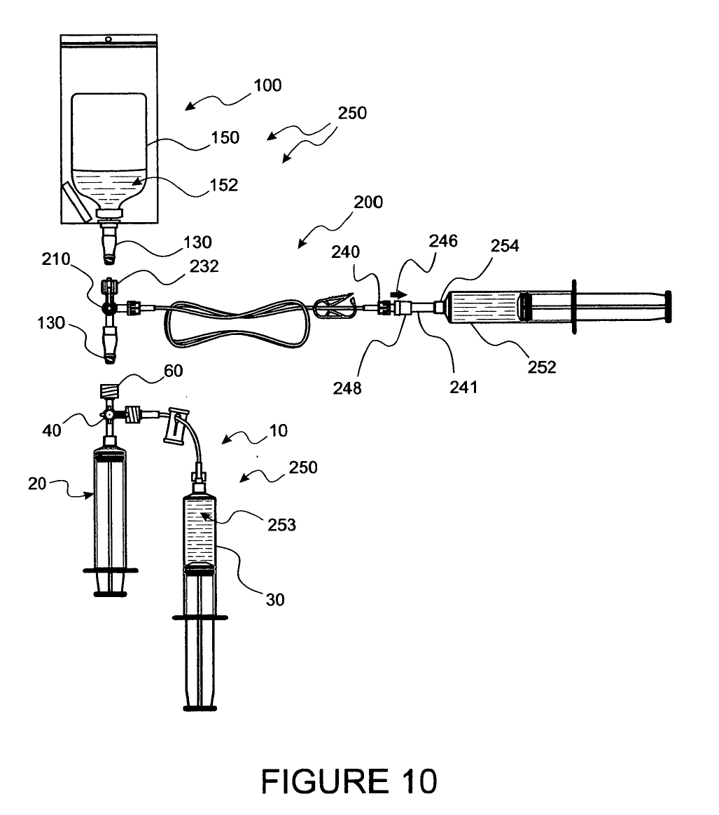

To provide a filling system (generally numbered 250 for assemblies 10 or 10′ assembled kits 100 and 200 are gathered as seen in FIG. 10 (with assembly 10 being used as an example). Note that vial 150 provides a source for fluid 152 to be transferred to syringe 20. Note also that flush syringe 30 is pre-filled with flush solution. A waste receiving syringe 252, is affixed via a female/female coupler 241 to distal end female/male luer connector 240 and intermediate one-way valve 248.

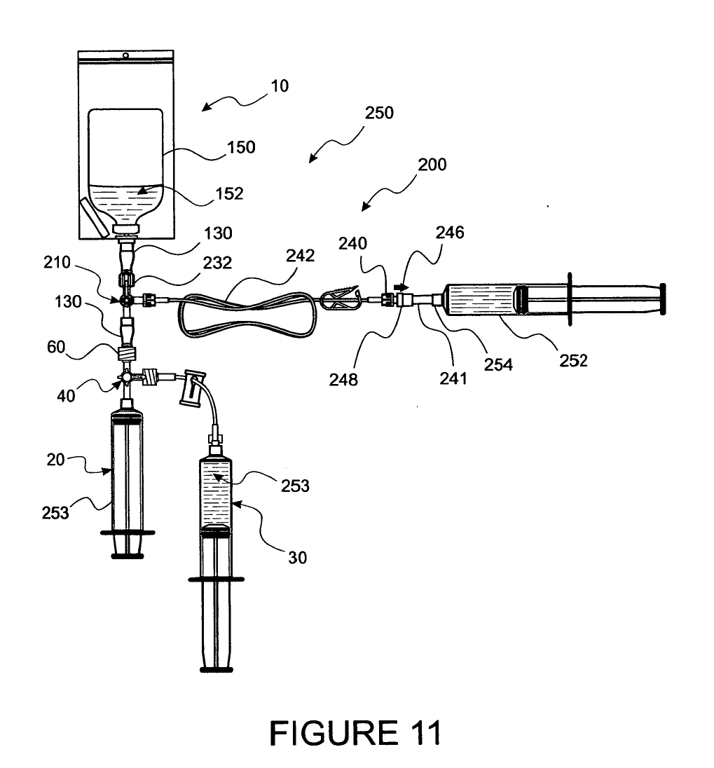

In preparation for filling, syringe 20 is used to draw a predetermined measurement of liquid 152 from vial 150. Prior to drawing from vial 150, a desired measurement of gas is preferably drawn into syringe 20 prior to connecting fitting 60 to needleless connector 130. Then fitting 60 is securely, but releasibly affixed to needleless connector 130. To complete assembly of filling system 250, fitting 232 is also securely, but releasibly, affixed to needleless connector 130. A completely assembled filling system 250 for assemblies 10 and 10′ is seen in FIG. 11.

As seen in FIG. 11, vial 150 initially has a volume of liquid available for delivery to a receiving vessel. In a preferred transfer mode, stopcocks 210 and 40 are adjusted to open a pathway from vial 150 to syringe 20. Preferably, using negative pressure technique, gas in syringe 20 is exchanged for a measured amount of liquid 152 in vial 150, syringe 20 being used as the measuring instrument. Once the predetermined amount of liquid 152 has been transferred to syringe 20, the pathway through stopcock 210 to vial 150 is closed and a pathway from syringe 20 into micro-bore tubing 242 and waste syringe 252 (which in this case acts as a waste vessel or container) is opened. Stopcock 40 is then adjusted to permit flush liquid 253 to be dispensed from syringe 30 through the site of a connection between fitting 60 and interconnected needleless connector 130. Three things should be noted. First system 250 is totally closed during fluid transfer. Second, when a disconnection is made between fitting 60 and needleless connector 130 after flushing, effectively, only flush solution is exposed at the disconnection sites. Third, a needleless connector 130 retards flow from superiorly connected flow paths until a subsequent connection is made by another luer fitting. For these reasons, assembly 10 (or assembly 10′) may be removed with safety of not exposing concentrated hazardous drugs at the disconnection site. Therefore fitting 60 may be capped to make filled assembly 10 or 10′ ready for transport to a site of use.

In this manner other assemblies 10 or 10′ may be affixed to needleless connector 130 and additional draws can be made from vial 150. At the time vial 150 is emptied, only a partial dose may be available to syringe 20. For this reason, it is expedient that empty vial 150 be exchanged for another vial containing more medication 152 to assure transfer of a complete dose to syringe 20. In any event, once use of a current vial 150 is complete for any reason, a pathway via stopcocks and 40 is opened, flush solution is delivered through the connected site between fitting 232 and needleless connector 130 to permit fitting 232 and needleless connector 130 to be disjoined and assembled kit 100 be delivered to a waste site per institutional protocol. In this manner, components associated with assembled kit 200 can be used for multiple dose acquisition from one or more vials 150 for delivery of measured medicine doses to syringe 20 of an assembly 10 or 10′.

### IV Container Filling Convenience Kit (Dual Stopcock)

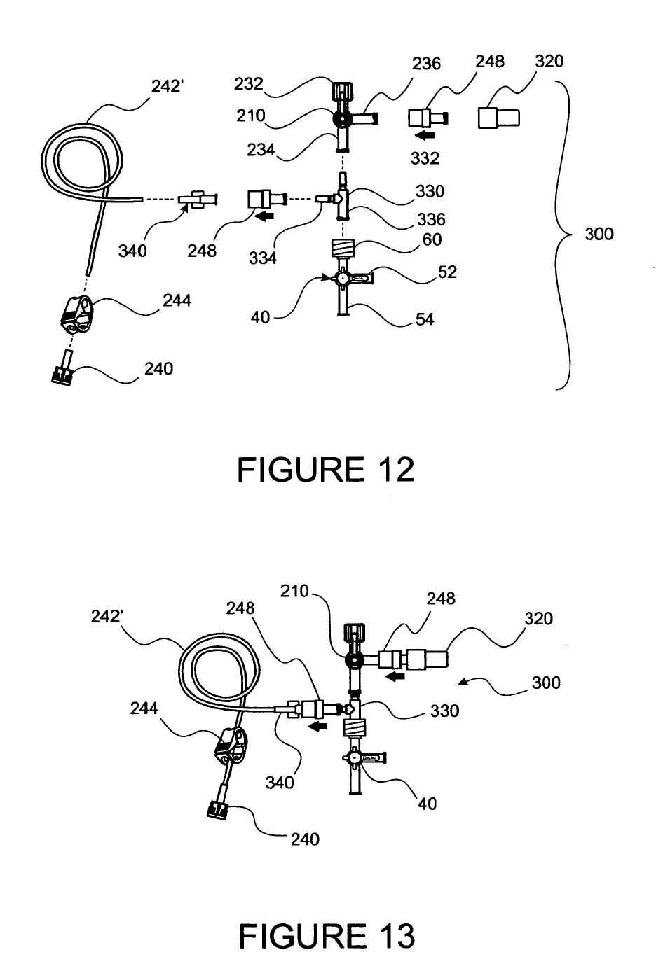

Filling assemblies 10 and 10′ are but one application of the instant invention. Parts for another convenience kit 300, according to the present invention, for use in filling a receiving IV vessel or container are seen in exploded format in FIG. 12 and further seen in FIGS. 13-16. Parts of convenience kit 300 comprise a stopcock 210; a one way valve 248; an air filter 320, a “T” connector 330 having two male connectors, individually numbered 332 and 334, and one female connector 336; a stopcock 40; a second one way valve 248, female luer-to-tube fitting 340; a length of micro-bore tubing 242′; a clamp 244; a male luer-to-tubing fitting 240. Parts not mentioned supra may be acquired for “T” connector 330: Qosina part number 88216; for one way valve 248: Filtertek part number 8300216; and for filter 320: BBraun part number 55003300. Part 340 is a standard female luer part that is well known by those skilled in medical assembly art and widely available world wide.

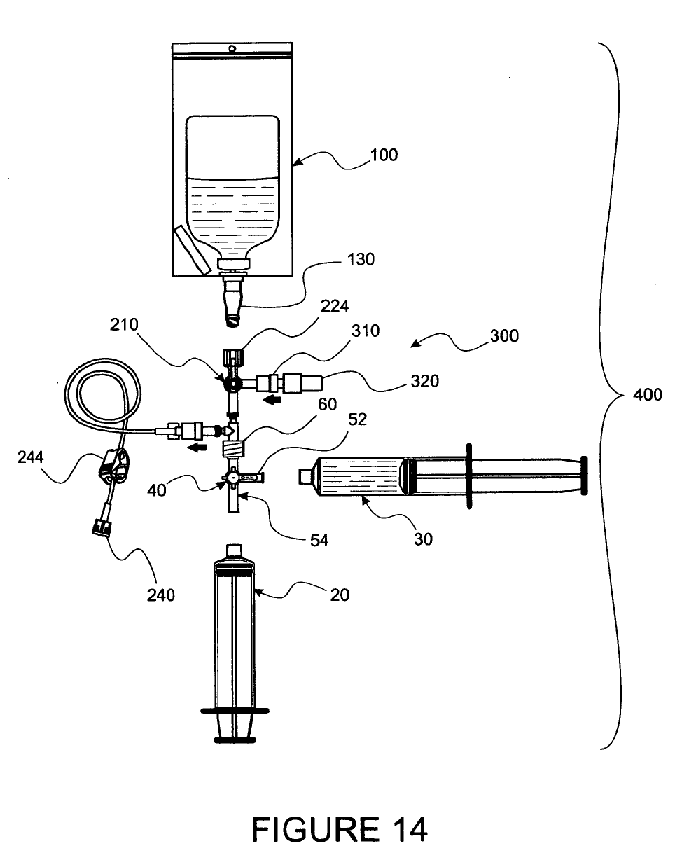

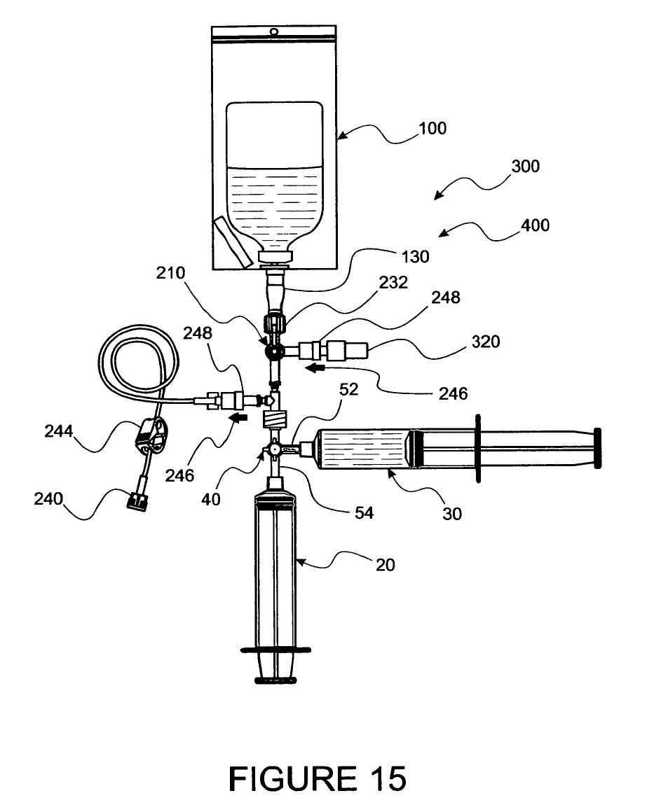

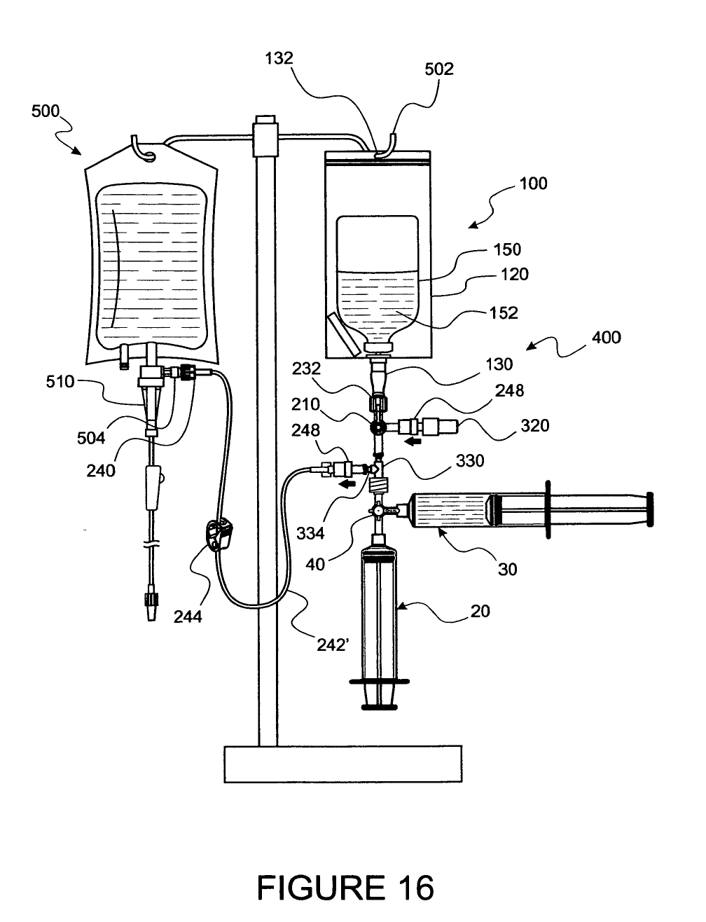

Parts of convenience kit 300, except clamp 244 which is disposed about tubing 242′, are unitized as seen in FIG. 13. Similar to system 250 seen in FIG. 10, assembled kits 100 and 300 are gathered as seen in FIG. 14 for assembly of filling system 400. A dose measurement syringe 20 and a pre-filled flush syringe 30 are also gathered. As seen in FIG. 15, assembled kits 100 and 300 are securely, but releasibly joined, by affixing male luer fitting 232 to needleless connector 130, syringe 20 is securely affixed to female luer fitting 54 and pre-filled syringe 30 is securely affixed to female luer fitting 52. Once so assembled filling system 400 is prepared to fill a selected IV receiving container or vessel 500 as seen in FIG. 16. Note that bag 120 is hung for facile user interface via hole 132 upon a hook 502. It may be further noted that such a hook may be a part of a ring stand, a hook provided within a hooded chamber or other work station site. End male luer fitting 240 is securely, but releasibly affixed to a side port 504 of a bag spike 510 for dispensing into container 500 to provide a closed system for transferring there into a dose of medication 152 from vial 150. Bag spikes with dry spike connections are widely available. Such a bag spike, having a part number of CH-12, may be acquired from ICU Medical. Note that, once so joined assembled filling system 400 and container 500 form a closed system for fluid transfer.

In this case, syringe 20 is used as a measuring instrument to interchange fluids and draw a predetermined volume of medication 152 from vial 150. For initial gas insertion into syringe 20 for fluid exchange of medication 152 within vial 150, a pathway is established via stopcock 210 for a pathway from filter 320 to stopcock 40. Further, a pathway is established through stopcock 40 to syringe 20. In this manner, a measured amount of exchange gas (air) can be drawn into syringe 20. It should be noted that clamp 244 may be used to close tube 242′ so that no fluid is inadvertently dispensed through tube 242′ even though such should not occur due to desired negative pressure vial and gas acquisition techniques should be followed when transferring fluids from filter 320 and into and out of vial 150.

Following gas insertion into syringe 20, stopcock 210 is switched to provide a pathway from syringe 20 to vial 150 for a gas/liquid exchange. Once gas and liquid have been exchanged to provide a desired measurement of liquid medication in syringe 20, stopcock 210 is switched to block all, above ambient pressure, pressurized exchange of fluid through stopcock 320. Note that one way valve 248 affixed to filter 320 blocks such flow.

To deliver the measured bolus of liquid resident in syringe 20 into container 500, stopcock 40 being already switched to provide a pathway to side port 504 and if clamp 244 is clamped to close tubing 242′, it is released. With pathways so established, liquid measured in syringe 20 is dispensed into container 500. To clear the connection between fitting 240 and side port 504, stopcock 40 is switched to open a pathway from pre-filled syringe 30 and side port 504. Sufficient flush liquid is purged through fitting 240 and side port 504 to assure substantially only flush liquid exposure when connection between fitting 240 and side port 504 is broken. Note, side port should have a needleless or like connector which retards flow from container 500 when a disconnection is so made.

Once disconnected, container 500 is separated from filling system 400 and prepared for transport and further use according to institutional protocol. If fitting 240 is not directly connected to another container 500 for a subsequent filling procedure, fitting 240 should be capped.

If there is need to change vial 150 of kit assembly 100, stopcock 40 and stopcock 210 are switched to provide a pathway from pre-filled syringe 30 and the releasible connection between fitting 60 and needleless connector flushed. Once sufficiently flushed so that separated portions at the disconnected site exposes substantially only flush solution, assembly 100 can be displaced from assembly 400 with safety. Then, according to institutional protocol, each part can be preserved or delivered to waste according to institutional protocol.

### Vented Vial Adapter Convenience Kit

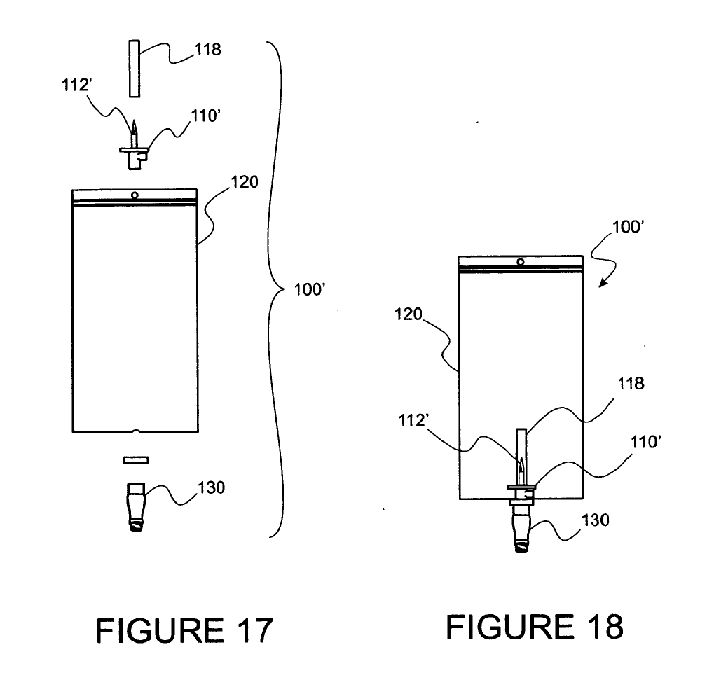

Reference is now made to FIGS. 17 and 18 wherein a vial shrouding and supporting convenience kit 100′ is seen. Convenience kit 100′ is like convenience kit 100 except that a vented vial adapter 110′ is provided in place of vial adapter 110, seen in FIGS. 3 and 4. By providing a vented vial adapter which relieves pressure and replaces fluid as liquid is drained from a vial (e.g. vial 150) steps required to use a measurement syringe (e.g. syringe 20) to replaces liquid drained from the vial are not required. Further, devices which permit pathway switching for delivery of gas to the measurement syringe are also not required. In such a case, care must be taken to provide sufficient volume of gas inside a closed and sealed shrouding bag 120. Parts of convenience kit 100′ are seen in exploded format in FIG. 17 and joined for kit packaging in FIG. 18.

### Ganged Delivery Convenience Kit

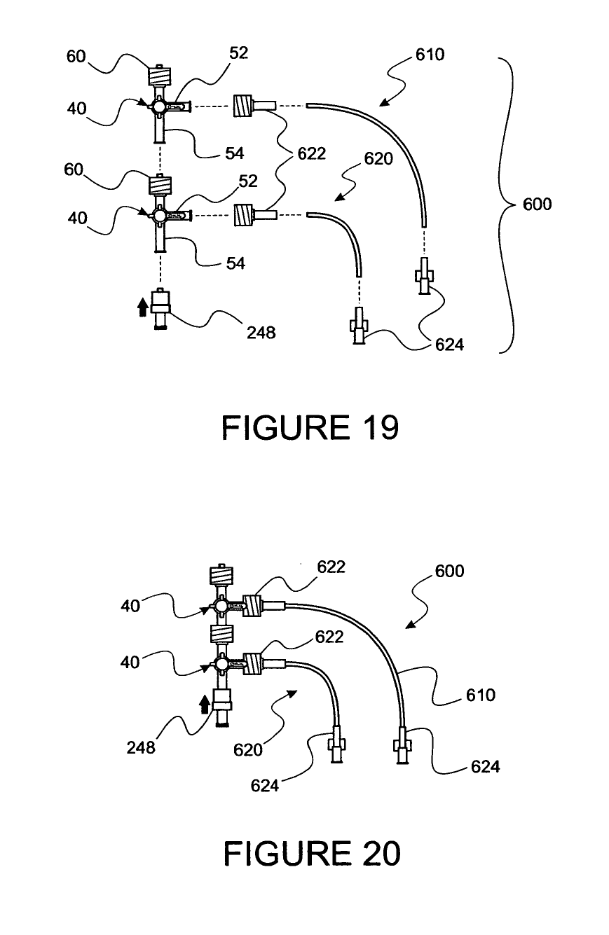

In some medical procedures, it is desirable to provide a series of two to three doses of different drugs to a patient in succession. Parts which may provide a set of ganged delivery syringes are seen in FIGS. 19-22. Seen in an exploded view in FIG. 19 are parts for a two dose kit 600. Parts for dose kit 600 include a first syringe pathway linkage 610 with a stopcock 40; a male luer to tubing fitting 622, a short length of micro-bore tubing and a female luer to tubing fitting 624 and a one-way valve 248. Parts of the first syringe pathway 610 are repeated to provide a second pathway linkage 620. Parts for kit 600 are joined and unitized as seen in FIG. 20.

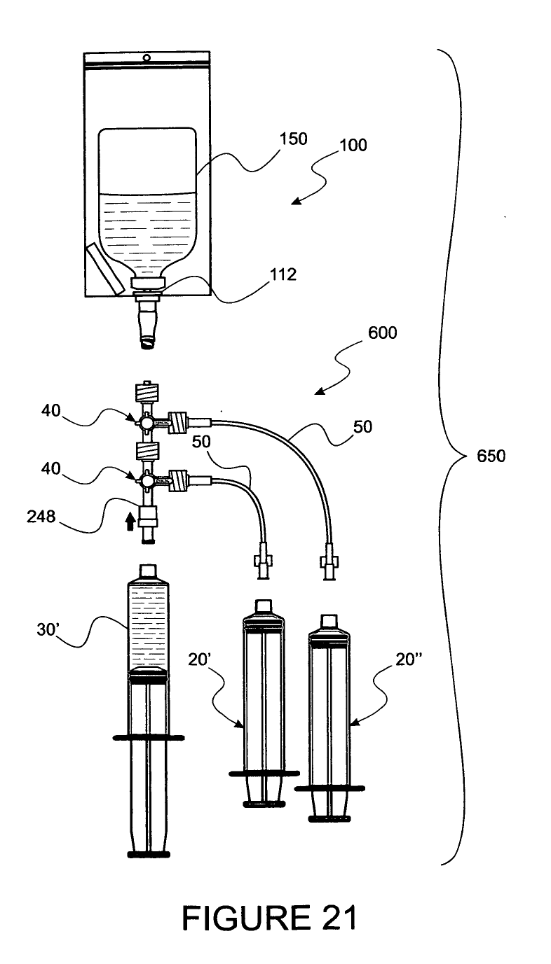

Parts 650 which are assembled for use of kit 600 in a syringe filling procedure are seen in exploded format in FIG. 21. Gathered for such an assembly of parts 650 are a convenience kit 100 with a vial 150 in place and pierced by a vial adapter spike 112 (not shown, but seen in FIGS. 3 and 4), ganged kit 600 and a plurality of syringes, referenced by application as one flushing syringe 30′ and two dose measuring and delivering syringes 20′ and 20″. Note that each assembly containing one stopcock 40, tubing 50 and other associated connectors is substantially the same as a similar assembly for a convenience kit associated with assembly 10 (see FIG. 1). Note, also, that such an assembly can be added to parts of kit 600 to add an additional dose syringe for a three syringe ganged assembly.

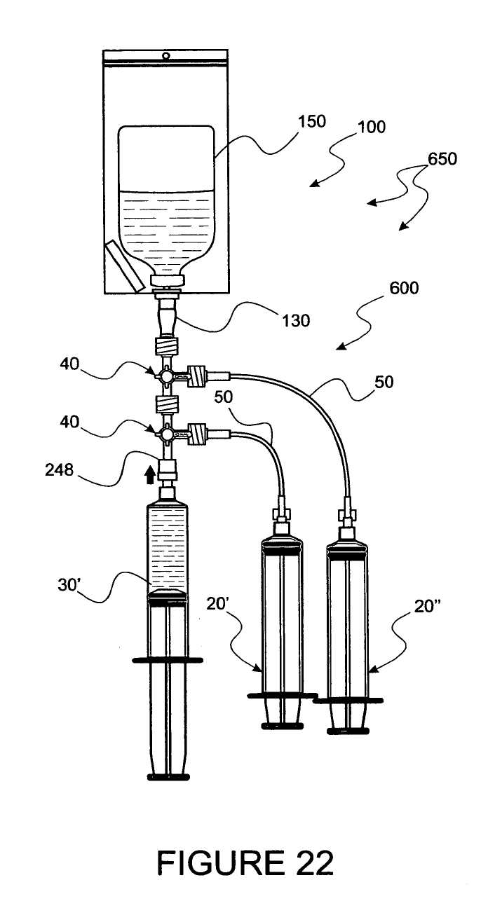

An assembled two syringe ganged assembly 650 comprising a completely assembled convenience kit 100 with a vial 150 which holds but a single dose, a convenience kit 600 and syringes 30′, 20′ and 20″ is seen in FIG. 22. So assembled, inferiorly disposed stopcock 40 is connected through one way valve 248 to syringe 30′ to assure fluid only flows from syringe 30′, in this case syringe 30′ being used as a flush syringe. In use, one of the stopcocks (for example inferiorly disposed stopcock 40) is switched to provide an open pathway from syringe 20′ to vial 150. Prior to opening such a pathway, it is preferred to dispose a volume of gas which is less but otherwise substantially the same as the volume of liquid to be drawn from vial 150. Upon opening the pathway, a predetermined volume of liquid initially resident in vial 150 is exchanged for gas in syringe 20′, syringe 20′ being the measuring instrument for determining amount of liquid volume exchange. Once the desired amount of liquid is disposed in syringe 20′, the inferiorly disposed stopcock 40 is switched to provide a pathway from syringe 30′ to vial 150 and a predetermined amount of flush liquid (e.g. 2 ml) is flushed into vial 150 to clear the connection surfaces between superiorly disposed stopcock 40 and needleless connector 40. At this point assembly 600 can be separated from kit assembly 100.

Of course, another medicant from another assembly 100 (vial 150) must be used as a source for a liquid dose in syringe 20″. Therefore, a different assembly 100 is provided with another vial 150 having the desired medicant which is to be displaced into syringe 20″. Effectively, the procedure for transferring medicant into syringe 20′ is repeated for syringe 20″, excepting, of course, switching superiorly disposed stopcock 40, rather than inferiorly disposed stopcock 40, to controllably open and close pathways between syringe 20″ and vial 150.

Once both syringes 20′ and 20″ are filled with measured amounts of liquid, the connection between needleless connector 130 and superiorly disposed stopcock 40 is purged. Kit assembly 100 is disconnected and disposed of per institutional protocol and the rest of the assembly (assembly 600 and syringes 20′, 20″ and 30) are prepared according to institutional protocol for delivery to a site of use.

IV Container Filling Convenience Kit (with 3-Way Stopcock)

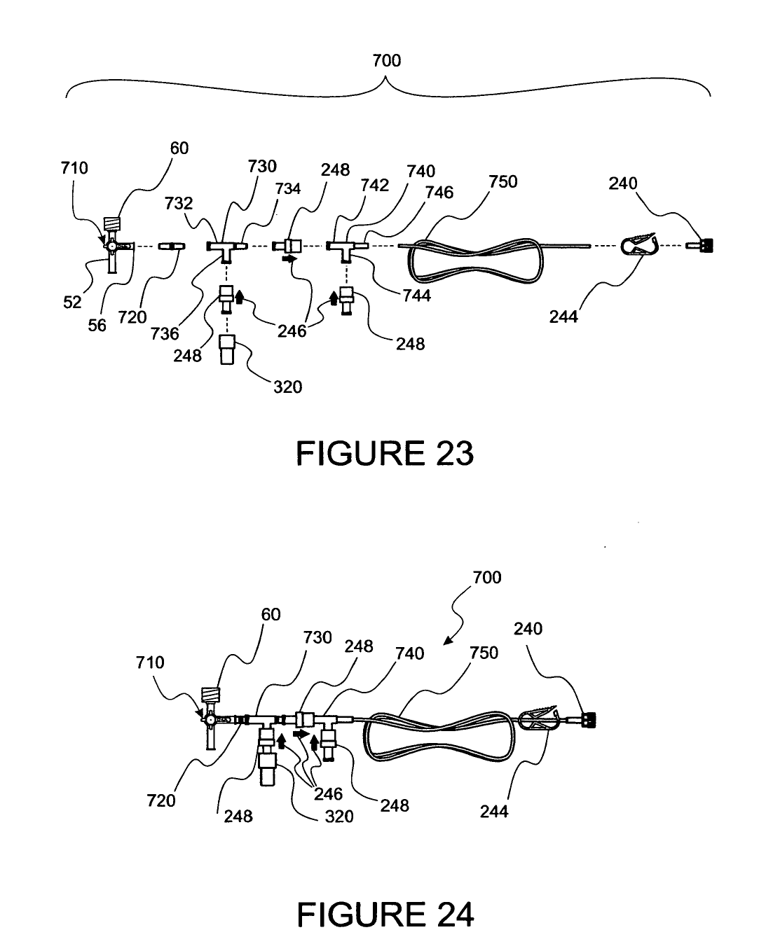

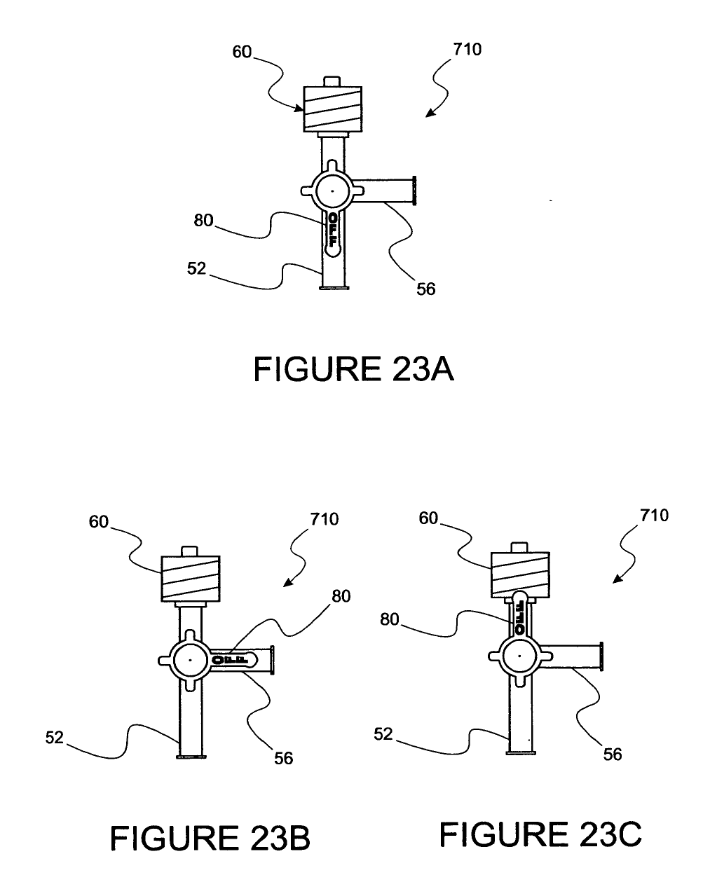

Reference is now made to FIGS. 23-26. Parts for a convenience kit 700 are seen in FIG. 23. Parts of kit 700 include but one 3-way stopcock 710 having one male luer fitting 60, an in-line female fitting 52 and a side port female fitting 56. Note that these fittings are similar in form and function to fittings 52, 56 and 60 of stopcock 40 (See FIG. 1), however, stopcock 710 is a 3-way stopcock rather than a 2-way stopcock. It should be noted that fitting 60 is a needleless connector compatible fitting as disclosed supra and disclosed in more detail hereafter.

Other parts in convenience kit 700 include a male/male coupler 720; a “T” connector 730 having a female luer fitting 732, and in-line male luer fitting 734 and a medially disposed female luer fitting 736; a first one-way valve 248 disposed for connection to fitting 736; a preferably 0.2 micron filter 320, a second one-way valve 248 disposed to connect to fitting 734; a second “T” connector 740 having a female luer fitting 742, a medially disposed female luer fitting 744 and a male tubing fitting 746; a length of micro-bore tubing 750 (currently having a preferred length of 15 inches); a clamp 244 disposed about tubing 750; and, finally, a fitting 240 for connecting to a receiving container, for being affixed to the distal end of micro-bore tubing 750.

Parts not identified for commercial acquisition, supra, which may be utilized as part of kit 700 are a 3-way stopcock having a male fitting which is compatible with a needleless connector, specially made for Applicants by Smith Medical and a male/male coupler, such as part number MLRC-9, available from Value Plastics. “T” connectors, micro-bore tubing and associated fittings are generally commercially available.

Parts seen in FIG. 23 are interconnected and the majority of parts unitized as seen in FIG. 24. It should be noted, however, that filter 320 is only securely, but removably affixed to associated one way valve 248. As disclosed hereafter, filter 320 is removable to permit a syringe to be affixed for purposes of dispensing liquid for flushing and reconstitution of dry contents in an associated vial 150 (see FIG. 10).

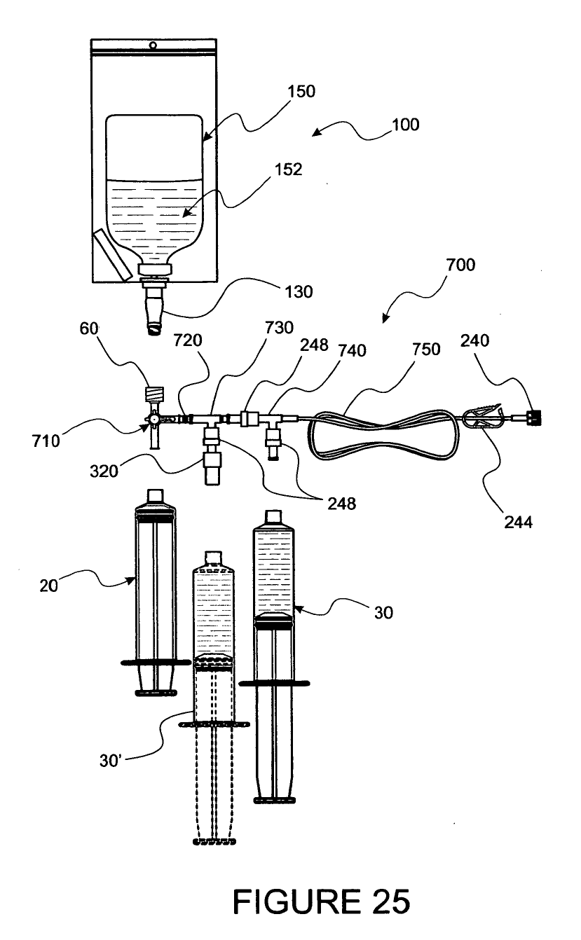

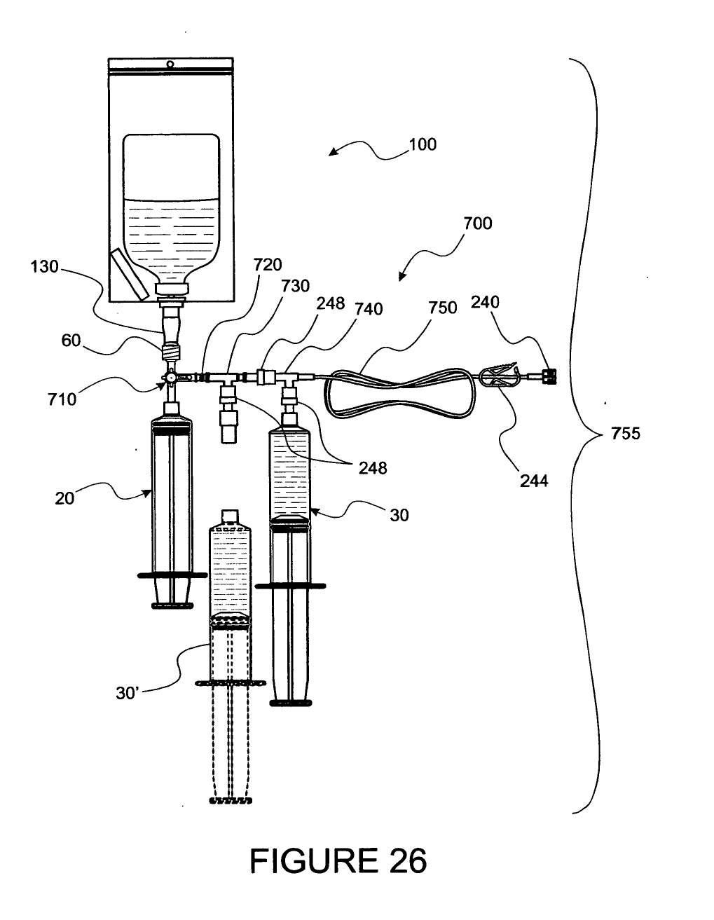

Two convenience kits 100 and 700 and an associated set of syringes 20, 30 and 30′ may be collected as seen in FIG. 25 in preparation for transferring fluid 152 from a vial 150 to a receiving container. Kits 100 and 700 are seen interconnected in FIG. 26 to form filling system 755. Note that syringes 20 and 30 are also connected, but syringe 30′ remains unattached. Syringe 30′ is reserved for use in dispensing liquid into vial 150 through the one-way valve seen affixed to filter 320.

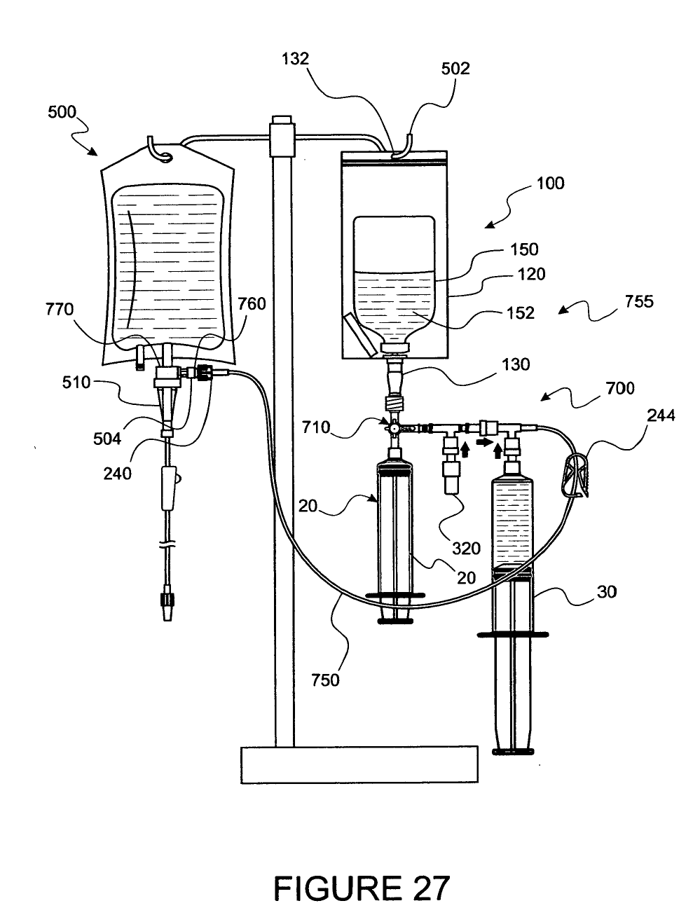

Fitting 240, affixed to the distal end of micro-bore tubing 750, is further connected to a selected IV receiving container or vessel 500 as seen in FIG. 27. Vessel 500 is simply representative of IV containers, in general. Such vessels may be bags, elastomeric balls, syringes or other medicine delivery receptacles. Note fitting 240 connected to a securely affixed but disconnectable port 760 of a bag spike 770. Note also, that the entire filling apparatus, including kit assembly 100 and kit assembly 700 and receiving vessel 500, is closed to the external environment until either a connection to needleless connector 130 or port 760 is broken.

In kit 700, stopcock 710 is used as the primary pathway selector and controller. Three possible pathway selecting positions of a rotary handle 80 (see FIG. 1 also) are seen in FIGS. 23A-C. Note that rotating handle 80 in line with fitting 52 closes the port associated with fitting 52 and opens a pathway between fittings 56 and 60. If handle 80 is rotated to alignment with fitting 56, (see FIG. 23B), a pathway is opened between fittings 52 and 60. Similarly, if handle 80 is rotated to alignment with fitting 60, a pathway is opened between fittings 52 and 56.

Thus, to fill syringe 20 with a predetermined volume of liquid from vial 150, with syringe 20 being used acting as a measuring instrument, stopcock 710 handle 80 is displaced as seen in FIG. 23B. A predetermined measure of liquid is drawn into syringe 20. Thereafter, handle 80 is rotated to be aligned with fitting 60, as seen in FIG. 23C for delivery of the predetermined measure through micro-bore tubing 750 to vessel 500. When a full dose has been delivered into vessel 500, a flush solution from syringe 30 through “r” connector 740 is dispensed through bag spike 770, permitting fitting 240 to be disconnected from bag spike 770 and only essentially flush solution resident on exterior exposed surfaces.

Drawing liquid from vial 150 necessarily creates a negative pressure therein. Note that rotating handle 80 into alignment with fitting 52 (see FIG. 23A) opens a pathway from filter 320 to vial 150 thus permitting relief from the negative pressure. Note also that, in this same handle 80 position, a pathway for flushing the connection between fitting 60 and associated needleless connector 130 is provided. This pathway is used for flushing by connecting a syringe 30′ (see FIG. 26) to a one-way valve 248 from which filter 320 is disconnected. (Note that clamp 244 should be used to close tubing 750 during this action) Thereby, should a vial 150 be emptied, connections to vial kit assembly 100 are flushed, permitting disconnection of the used kit assembly 100 while exposing essentially only flush solution at the disconnection site and, if desired, connecting a replacement vial kit assembly 100 to kit assembly 700 to continue with a desired medicine transfer procedure.

In this manner multiple doses may be drawn from a single vial. As well, multiple vials may be used to provide a single dose.

### Needleless Connector Compatible Stopcock Male Fittings and Interfaces

The following paragraphs relating to stopcock fittings and interfaces has been copied, in large part, from U.S. patent applications from which this U.S. patent application continues-in-part. Material contained in the cited following paragraphs is germane to each stopcock having an associated male luer fitting which interfaces with a needleless connector for reasons cited hereafter. For this reason, the following should be seriously considered when interconnecting a male luer fitting of a stopcock with a needleless connector because the majority of stopcocks currently commercially available may not couple to produce a reliably open flow path when connected to a needleless connector, for reasons disclosed in detail hereafter.

Commonly, needleless fittings are currently used as ports for IV sets affixed to patient catheters and contemporary vial adapters. These fittings have been designed to interface with male syringe luer fittings, such as luer fittings 892 and 892′, affixed to syringes 20 and 30, respectively, see FIG. 1. By standard specifications, male luer fittings have common outside diameters and frustoconical shapes. However, a review of inside diameters of male luer fittings reveals a marked difference between syringe male luer fittings and male luer fittings found on contemporary commercial stopcocks. Exemplary stopcock luer fittings are seen in FIGS. 1A-1D.

A stopcock 40 affixed to a tubing set 50 is seen in FIGS. 1A and 1B. Stopcock 40 and tubing set 50 are magnified in FIG. 1B for clarity of presentation with tubing set 50 is truncated. As seen in FIG. 1B, a proximally disposed luer fitting 60 has a male luer part 893 surrounded by a luer lock 894. As stated supra, luer part 895 has an outside surface 898 having a standard luer diameter and frustoconical shape, and, to meet requirements of interfacing with needleless connectors, has a medially disposed through bore hole 896.

Stopcock 40 is further magnified in FIG. 1D for additional clarity of presentation. To aid in understanding a basic difference between stopcock 40 and PRIOR ART stopcocks. An example of a PRIOR ART stopcock 40′ is provided in FIG. 1C for comparison. Attention is drawn to male luer part 60 of stopcock 40 in FIG. 1D and a similar male luer part 60′ of stopcock 40′ in FIG. 1C. Note that stopcock 40 has a through bore hole 896 which is relatively smaller than a through bore hole 896′ of stopcock 40′ (see FIG. 1C).

Thus, associated proximal luer face 897, disposed between outside surface 898 and through bore hole 896 is larger in surface area than an outside surface 898′, disposed between outside surface 895′ and through bore hole 896′ of stopcock 40′. Generally, in the past, it is believed that through bore hole 896′ in contemporarily commercially available stopcocks has been defined by draft specifications associated with injection molding. These draft specifications have resulted in the relatively larger size of bore hole 896′. It should be noted that such luer faces are circumferentially defined by outside surfaces having a smallest diameter of approximately 0.150 inches. Such is also true of stopcocks 40 and 40′.

However surface area of syringe luer faces are further defined by the through hole, similar to luer hole 896 of stopcock 40. Diameter of such a syringe through hole 896 is approximately 0.080 inches. Notably, diameter of an exemplary through hole 896′ for stopcock 40′ is approximately 0.120 inches. Note that a 0.120 inch diameter through hole yields a luer face width of about 0.015 inches while a 0.080 inch diameter yields a luer face width of about 0.035 inches. Such a difference in thickness of a luer face is a significant determinant in providing a reliable interface to a needleless connector which has been designed for use with syringe luer fitting dimensions. It is for this reason that stopcock 40 has a significantly smaller through hole diameter than stopcock 40′. Such a decreased size in luer diameter may be achieved by a change in mold design or by affixing a tube having a desired through hole diameter into a larger through hole, such as through bore hole 896′. For purposes of reference, such a stopcock male connector, having a bore hole and luer face thickness similar in dimension and function to a syringe luer connection, is further referenced herein as having a needleless compatible connector interface. Of course, such an interface may also be achieved by adding a female/male coupler to a stopcock male luer connection, such as male luer part 60′, if the female/male coupler has appropriate connecting geometry to provide a needleless compatible connector interface. While such a coupler addition is within the scope of the present invention, such is not preferred as addition of such a coupler adds undesirable cost and dead space.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. Apparatus for transferring contents of a medical vial through a closed system which remains closed throughout such transfer and which provides an associated flush which is an inherent part of the system such that each site upon disconnection is flushed prior to such disconnection to expose substantially only flush solution to the external environment, said apparatus comprising:
a vial adapter kit assembly comprising:
a shroud comprising an entry port through which a vial can be inserted for protective enclosure by the shroud prior to perforating a pierceable septum of the vial for the purpose of displacing contents from the vial, said shroud comprising a closure for the entry port such that the vial is effectively fully contained within the shroud and further comprising material which is substantially impervious to fluids and which is collapsible and expandable, yet sufficiently non-elastic to provide support for the vial within the shroud;
a vial adapter affixed to the shroud, said shroud further comprising an impeded opening where through a portion of the vial adapter is exposed from the shroud and disposed for transferring fluid from shroud and vial to a closed, switchable pathway controlling subsystem, said vial adapter comprising a spike, disposed within the shroud, for perforating the vial septum; and
a needleless connector affixed to the exposed portion of the vial adapter;
a closed, switchable pathway controlling subsystem comprising a fluid switching device securely, but releasibly affixed to the needleless connector, the switching device, in a first state, selectively provides a course for fluid flow from the needleless connector to a fluid displacement and measuring instrument for transferring predetermined amounts of liquid from the vial and, in a second state, provides a course for fluid flow to an exterior connector which, during liquid transfer, is affixed to a liquid receiving container which is later separated from said apparatus for delivery to a site of use; and
an associated source of flush solution, isolated during transfer of liquid from the vial but disposed in switchable communication with each site where a disconnection can be made, via the pathway controlling subsystem, such that, prior to disconnection from each such site, flush solution is displaced through each such site before disconnection, thereby permitting liquid exposed by disconnection to be substantially flush solution.

2. Apparatus according to claim 1 wherein said shroud comprises a plastic bag having a zipper closure.

3. Apparatus according to claim 2 wherein said bag comprises a superiorly disposed hole whereby the apparatus may be hung from a hook such that a user's hanks are freed thereby from maintaining stability of the vial during fluid transfer operations.

4. Apparatus according to claim 1 wherein said apparatus further comprises a predetermined and controlled pathway for gas influx into the apparatus whereby a volume of gas can be displaced into the vial to replace liquid withdrawn therefrom.

5. Apparatus according to claim 1 wherein said apparatus comprises two flushable and separable parts, a first part comprising said needleless connector and said shroud and a second part comprising the closed switchable pathway.

6. Apparatus according to claim 5 wherein said first part comprises said needleless connector affixed to the exposed portion of the vial adapter to thereby provide a site for disconnection at the end of the needleless connector distal from the vial adapter and said second part comprises a stopcock comprising a needleless compatible male luer interface whereby the closed switchable pathway is securely, but separably, affixed to the needleless connector for fluid communications between the two parts.

7. Apparatus according to claim 1 wherein said source of flush solution comprises a pre-filled flush syringe.

8. Apparatus according to claim 1 wherein said closed switchable pathway controlling system comprises pathway selection components for delivering flush solution through the site of connection between the needleless connector and needleless connector compatible male luer interface.

9. Apparatus according to claim 8 wherein said source of flush solution comprises a one-way valve for assuring no back-flow contamination of fluid into the flush syringe.

10. Apparatus according to claim 1 wherein said measuring instrument comprises a conventional syringe.

11. Apparatus according to claim 1 wherein the closed, switchable pathway controlling subsystem further comprises a 3-way valve, having a needleless connector compatible male luer fitting interface, and 3-way valve associated selectable fluid pathways comprising:
(a) a pathway between a gas filter and the associated vial and a pathway between a flush syringe and an associated vial when the filter is removed and replaced by a pre-filled flush syringe;
(b) a pathway between a measuring instrument and the associated vial; and
(c) a pathway between the measuring instrument and a receiving IV container.

12. A method for transferring contents of a medical vial through a closed system which remains closed throughout such transfer and which provides an associated flush which is an inherent part of the system such that each site where a disconnection is made to permit fluid exposure to external environment is flushed prior to such disconnection to present substantially only flush solution at the exposed sites, said method comprising the steps of:
(a) providing:
(I) a closeable and sealable shroud for protective enclosure of a vial from which medication is transferred;
(II) a vial adapter affixed to the shroud, said shroud further comprising a barrier where through a portion of the vial adapter is exposed from the shroud and disposed for transferring fluid from shroud and vial, said vial adapter comprising a spike, disposed within the shroud, for perforating a pierceable vial septum;
(III) a needleless connector affixed to the exposed portion of the vial adapter;
(IV) a closed, switchable pathway controlling subsystem comprising a fluid switching device which, in a first state, provides a course for fluid flow from the exposed portion of the vial adapter through the needleless connector to a fluid displacement and measuring instrument for transferring predetermined amounts of liquid from the vial, said fluid switching device, in a second state, providing a course for fluid flow to an exterior connector which during liquid transfer is affixed to a liquid receiving container which is later separated for delivery to a site of use; and
(V) an associated source of flush solution, isolated during transfer of liquid from the vial but disposed in selectable switchable communication with each site where a disconnection is to be made such that, prior to disconnection from each such site, liquid is flushed through that site permitting liquid exposed by disconnection to be substantially flush solution;
(b) following institutional protocol for handling a selected vial, displacing the selected vial into said shroud and enclosing and sealing the vial within said shroud;
(c) as needed, removing any protective covering from the spike of said vial adapter and perforating a pierceable septum of the vial with the spike whereby fluid within the vial is made accessible through said vial adapter without direct manual contact with vial adapter or vial;
(d) drawing a predetermined volume of liquid from the vial into the measurement instrument;
(e) dispensing the predetermined volume of liquid from the measurement instrument into the receiving container;
(f) providing a communicating pathway between the source of flushing liquid and the exterior connector through which liquid was dispensed to the liquid receiving container; and
(g) flushing through the exterior connector and thereafter disconnecting the exterior connection associated with the exterior connector to thereby only expose flushing solution to the environment exterior to the otherwise closed system.

13. The method according to claim 12 comprising a further step (VI) of providing a switchable pathway from the source of flush solution to the needleless connector and needleless compatible male luer fitting interface for a connecting stopcock, which is part of the pathway controlling subsystem, whereby the site of disconnection between the needleless connector, vial adapter and pathway controlling subsystem is flushed prior to disconnection to thereby provide for separating the vial adapter and shroud from the pathway controlling subsystem for replacement thereof.

14. The method according to claim 13 comprising the following steps:
(h) flushing the needleless connector disconnection site;
(i) disconnecting the attached needless connector and associated vial adapter, shroud and vial for the purpose of replacement thereof; and
(j) affixing a different needleless connector and associated vial adapter, shroud and vial replacement to the needleless compatible male luer fitting interface for the stopcock.

15. The method according to claim 12 further comprising a step, associated with step (d), said step comprising:
(k) providing a predetermined switchable pathway for drawing gas into the apparatus whereby a volume of gas can be displaced into the vial to replace liquid withdrawn therefrom.

16. The method according to claim 15 comprising the following additional steps, associated with steps (d) and (k):
(l) displacing gas into the attached fluid measurement instrument before drawing the predetermined volume of liquid from the vial into the fluid measurement instrument; and
(m) exchanging liquid for gas in the measurement instrument to displace the predetermined volume of liquid from the vial into the measuring instrument.

17. The method according to claim 12 wherein step III comprises providing a syringe for the measuring instrument.

18. The method according to claim 12 wherein step IV comprises providing a syringe pre-filled with flush solution.

19. A vial adapter convenience kit for handling and transferring liquids from a medical vial with safety, said adapter kit comprising:
a shroud comprising an entry port through which a vial can be inserted for protective enclosure in the interior of the shroud prior to perforating a pierceable septum of the vial for the purpose of displacing contents from the vial, said shroud comprising a closure for the entry port such that the vial is effectively fully contained within the shroud and further comprising material which is substantially impervious to fluids and which is collapsible and expandable, yet sufficiently non-elastic to provide support for the vial within the shroud while permitting spiking of the vial via contact only with the exterior of the shroud;
a vial adapter affixed to the shroud, said shroud further comprising an inferiorly disposed opening having a gasket where through a portion of the vial adapter is exposed outside the shroud and, thereby, disposed for transferring fluid from the vial outside said shroud, said vial adapter comprising a spike, disposed within the shroud, for perforating the vial septum; and
said shroud further comprising a superiorly disposed hanging accessory whereby the shroud may be hung from a fixture to thereby orient the vial for dispensing and to provide support for vial adapter and vial.

20. A vial adapter according to claim 19 further comprising a needleless connector affixed to the exposed portion of said vial adapter, said needleless connector having a luer fitting which is substantially completely purged of resident liquid when flushed through a needleless compatible luer fitting interface affixed to a distal end thereof.
